(19) United States
(12) Patent Application Publication
(10) Pub. No.: US 2021/0342212 A1
(43) Pub. Date: Nov. 4, 2021

(54) METHOD AND SYSTEM FOR IDENTIFYING ROOT CAUSES

(71) Applicant: KPN INNOVATIONS, LLC., LAKEWOOD, CO (US)

(72) Inventor: Kenneth Neumann, Lakewood, CO (US)

(73) Assignee: KPN INNOVATIONS, LLC., LAKEWOOD, CO (US)

(21) Appl. No.: 17/365,866

(22) Filed: Jul. 1, 2021

Related U.S. Application Data
(63) Continuation-in-part of application No. 16/590,417, filed on Oct. 2, 2019, now Pat. No. 11,107,555.

(51) Int. Cl. G06F 11/07; G06N 20/00; G06F 40/20; G06N 3/02; G06F 3/16; G06N 5/02

(52) U.S. Cl. CPC G06F 11/079; G06N 20/00; G06N 5/02; G06N 3/02; G06F 3/167; G06F 40/20

(57) ABSTRACT

A system for identifying root causes, the system including a computing device designed and configured to receive a user input from a user client device, extract at least a symptom datum form the user input, extracting the at least a symptom datum includes being configured to generate at least a query using the user input, and generate the at least a symptom datum as a function of the at least a query, train a machine learning process with an expert input training set from an expert knowledge database wherein the expert input training set further includes prognostic data correlated to causal link data, configured to assign weights to the correlated data as a function of the at least a symptom datum, identify root causes as a function of the assigned weights and display the root causes to the user.

### CROSS-REFERENCE TO RELATED APPLICATIONS

This application is a continuation-in-part of non-provisional application Ser. No. 16/590,417 filed on Oct. 2, 2019 and entitled “METHODS AND SYSTEMS FOR IDENTIFYING A CAUSAL LINK,” the entirety of which is incorporated herein by reference.

### FIELD OF THE INVENTION

The present invention generally relates to the field of artificial intelligence. In particular, the present invention is directed to methods and systems for identifying at least a root cause.

### BACKGROUND

Accurate identification of root causes can be challenging. Frequently, practitioners are unaware of a root cause that may ultimately be attributing to a symptom. Further, this problem is exacerbated by the plethora of medical literature available that practitioners often lack adequate time to read and analyze.

### SUMMARY OF THE DISCLOSURE

In an aspect, a system for identifying root causes. The system includes a computing system, where the computing system is designed and configured to receive a user input for a user device; extract at least a symptom datum from the user input, where the extraction of the at least a symptom datum further includes computing system being designed and configured to generate at least a query using the user input and generate the at least a symptom datum as a function of the at least a query. The system further designed and configured to train a machine learning process with an expert input training set where the expert input training set further include prognostic data correlated to causal link data; assign weights to the correlated data as a function of the symptom datum; identify root causes as a function of the assigned weights; and display the root causes to the user.

In another aspect, a method of identifying root causes. The method includes receiving, by a computing device, a user input from a user client device; extracting, by the computing device, a symptom datum from the user input where extracting the symptom datum includes generating at least a query using the user input and generating the at least a symptom datum as a function of the at least a query. The method further includes training, by the computing device, a machine learning process with an expert input training set from an expert knowledge database where the expert input training set further includes prognostic data correlated to causal link data; assigning weights, by the computing device, to the correlated data as a function of the symptom datum; identifying, by the computing device, root causes as a function of the assigned weights; and displaying, by the computing device, the root causes to the user.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating a system for identifying a root cause;

FIG. 2 is a process flow diagram illustrating an exemplary embodiment of a method of identifying a root cause;

FIG. 3 is a block diagram illustrating an exemplary embodiment of a machine-learning database;

FIG. 4 is a block diagram illustrating an exemplary embodiment of an expert knowledge database;

FIG. 5 is an exemplary embodiment of a machine learning module;

FIG. 6 is an exemplary embodiment of a neural network;

FIG. 7 is an exemplary embodiment of a node of a neural network; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for identifying a root cause. In an embodiment, the system includes a computing device that receives a user input from a user client device, and extracts a symptom datum by using a parsing module and a language processing module. The system trains a machine learning process with an expert input training set form an expert knowledge database. The expert input training set includes prognostic data correlated to causal link data. The system correlates the at least a symptom datum to the expert input training set as a function of the machine learning process. The system assigns weights to the correlated data as a function of the at least a symptom datum, identifies a root cause as a function of the assigned weights and displays the root cause to the user.

Aspects of the present disclosure can be used to extract symptoms from a user natural language input. Aspects of the present disclosure can also be used to identify a potential root cause based on the symptoms provided by a user. This is so, at least in part, because the system is configured to utilize machine learning process to correlate the symptoms provided by a user with root causes associated with the same symptoms form an expert dataset.

Aspects of the present disclosure allow for providing a user with a potential root cause based on the symptoms provided, where the user may provide those symptoms through a plurality of mediums such an interactive form in their smartphones or a voice recording describing what they a feeling. Exemplary embodiments illustrating aspects of the present disclosure are described below in the context of several specific examples.

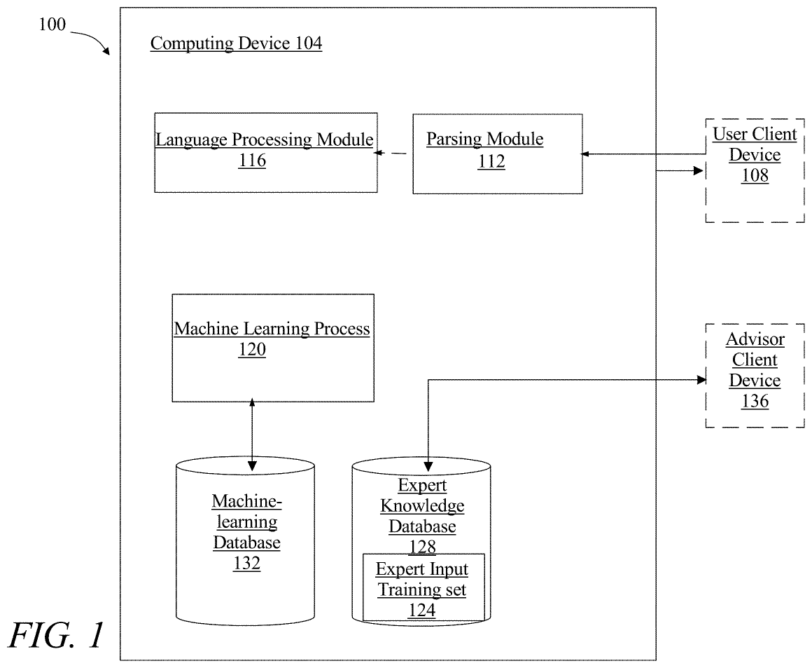

Referring now to FIG. 1, an exemplary embodiment of a System 100 for identifying a root cause is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device receives a user input from a user client device 108. Client user device may include, without limitation, a display in communication with computing device, display may include any display as described herein. User client device 108 may include an additional computing device, such as a mobile device, laptop, desktop computer, or the like. As a non-limiting example, user client device 108 may be a computer and/or workstation operated by a medical professional. In another non-limiting example, user client device 108 may be a computing device operated by a patient.

Still referring to FIG. 1, user client device 108 may include a graphical user interface (GUI). As described herein, a graphical user interface is a form of user interface that allows users to interact with the computing device 104 through graphical icons and/or visual indicators. The user may, without limitation, interact with graphical user interface through direct manipulation of the graphical elements. Graphical user interface may be configured to receive input through voice interaction. The user may, without limitation, interact with graphical user interface though video interaction. Graphical user interface may be configured to display a root cause, as described in detail below. As an example, and without limitation, graphical user interface may be displayed on any electronic device, as described herein, such as, without limitation, a computer, tablet, remote device, and/or any other visual display device.

With continued reference to FIG. 1, user client device 108 may include a graphical user interface having, without limitation, a form or other graphical element having data entry fields, wherein a user may enter an input. GUI may include data entry fields that allow for a user to enter free form textual inputs. GUI may provide drop-down lists, where users may be able to select one or more entries to indicate one or more user symptom datums. Without limitation, user client device 108 may include voice-to-text functionality, wherein user may enter an input through voice.

With continued reference to FIG. 1, computing device 104 is configured to extract at least a symptom datum from user input. A “symptom datum”, as used in this disclosure, is an element of data describing information relevant to human subject's state of health, including, without limitation, symptoms, conditions, prognoses, test results, concerns, reasons for a visit to a healthcare professional, personal stories and/or information concerning the human subject's interests, relationships to other people, informal and/or formal personal or health support groups or persons, orthe like. A symptom datum may include a current medical indicator. A “current medical indicator” as used in this disclosure, includes an element of data describing any subjective description of a current or future probable disease that a user is experiencing. Subjective descriptions may include any phenomenon a user may be experiencing including for example anxiety, pain, fatigue, tremor, headache and the like. A first user symptom datum may be apparent as indicating a particular condition and/or disease such as when a user experiences blood loss from a subcutaneous flesh would. A symptom datum may not be apparent as indicating a particular condition and/or disease such as when a user may experience tiredness due to a thyroid disease which a user may believe is due to being overly fatigued. A “disease” as used in this disclosure, includes an abnormal condition that negatively affects the structure and/or function of part of a human body. A disease may include a current disease diagnosed by a health professionalwho may be authorized by a particular health licensing board to diagnose disease and/or conditions such as for example a medical doctor, a doctor of osteopathy, a nurse practitioner, a physician assistant, a doctor of optometry, a doctor of dental medicine, a doctor of dental surgery, a naturopathic doctor, a doctor of physical therapy, a nurse, a doctor of chiropractic medicine, a doctorof oriental medicine, and the like. A disease may include a future probable disease identified by the presence of one or more predisposing factors. Predisposing factors may include genetic predispositions such as a genetic characteristic which influences the possible phenotypic development of a disease. For instance and without limitation, a genetic characteristic such as a mutation of breast cancer gene 1 (BRCA1) may predispose a user to have a predisposition to developcertain cancers such as breast and ovarian cancer. Predisposing factors may include behavior predispositions such as negative behaviors that may predispose a user to certain future probable diseases. For example, a negative behavior such as smoking may predispose a user to lung cancer while a negative behavior such as eating foods high in saturated fats may predispose a user to heartdisease.

With continued reference to FIG. 1, to extract the at least a symptom datum, the computing device 104 is configured to generate at least a query using the user input. In an embodiment, generating the at least a query using user input may include using a parsing module 112. At least a query, as used in this disclosure, is at least a datum used to retrieve text that will be incorporated in at least a textual output, where retrieval may be effected by inputting the at least a query into a data structure, database, and/or model, and receiving a corresponding output as a result, for example as set forth in further detail below. Parsing module 112 may generate at least a query by extracting one or more words or phrases from the input, and/or analyzing one or more words or phrases; extraction and/or analysis may include tokenization, in relation to a language processing module 116. A language processing module 116 may generate the language processing model by any suitable method, including, without limitation, a natural language processing classification algorithm; language processing model may include a natural language process classification model that enumerates and/or derives statistical relationships between input term and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMIs as used herein are statistical models with inference algorithms that that may be applied to the models. In such models, a hidden state to be estimated may include an association between an extracted word category of physiological data, a given relationship of such categories to prognostic labels, and/or a given category of prognostic labels. There may be a finite number of category of physiological data, a given relationship of such categories to prognostic labels, and/or a given category of prognostic labels to which an extracted word may pertain; an HMM inference algorithm, such as the forward-backward algorithm or the Viterbi algorithm, may be used to estimate the most likely discrete state given a word or sequence of words. Language processing module 116 may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

With continued reference to FIG. 1, parsing module 112 may utilize, incorporate, or be a language processing module 116 as described above. Language processing module 116 may be configured to map at least a user input to at least a query, using any process as described above for a language processing module 116. Extraction and/or analysis may further involve polarity classification, in which parsing module 112 may determine, for instance, whether a phrase or sentence is a negation of the semantic content thereof, or a positive recitation of the semantic content; as a non-limiting example, polarity classification may enable parsing module 112 to determine that “my feet hurt” has a divergent meaning, or the opposite meaning, of the phrase “my feet don't hurt.” Polarity classification may be performed, without limitation, by consultation of a database of words that negate sentences, and/or geometrically within a vector space, where a negation of a given phrase may be distant from the non-negated version of the same phrase according to norms such as cosine similarity.

Continuing to refer to FIG. 1, parsing module 112 may be configured to normalize one or more words or phrases of user input, where normalization signifies a process whereby one or more words or phrases are modified to match corrected or canonical forms; for instance, misspelled words may be modified to correctly spelled versions, words with alternative spellings may be converted to spellings adhering to a selected standard, such as American or British spellings, capitalizations and apostrophes may be corrected, and the like; this may be performed by reference to one or more “dictionary” data structures listing correct spellings and/or common misspellings and/or alternative spellings, or the like. Parsing module 112 may perform algorithms for named entity recognition. Named entity recognition may include a process whereby names of users, names of informed advisors such as doctors, medical professionals, coaches, trainers, family members or the like, addresses, place names, entity names, or the like are identified; this may be performed, without limitation, by searching for words and/or phrases in user database. For instance, parsing module 112 may identify at least a phrase, which may include one or more words, map the at least a phrase to at least a query element, and then assemble a query using the at least a query element. Mapping at least a phrase to at least a query element may be performed using any language processing technique described in this disclosure, including vector similarity techniques.

With continued reference to FIG. 1, parsing module 112 may extract and/or analyze one or more words or phrases by performing dependency parsing processes; a dependency parsing process may be a process whereby parsing module 112 and/or a language processing module 116 communicating with and/or incorporated in parsing module 112 recognizes a sentence or clause and assigns a syntactic structure to the sentence or clause. Dependency parsing may include searching for or detecting syntactic elements such as subjects, objects, predicates or other verb-based syntactic structures, common phrases, nouns, adverbs, adjectives, and the like; such detected syntactic structures may be related to each other using a data structure and/or arrangement of data corresponding, as a non-limiting example, to a sentence diagram, parse tree, or similar representation of syntactic structure. Parsing module 112 may be configured, as part of dependency parsing, to generate a plurality of representations of syntactic structure, such as a plurality of parse trees, and select a correct representation from the plurality; this may be performed, without limitation, by use of syntactic disambiguation parsing algorithms such as, without limitation, Cocke-Kasami-Younger (CKY), Earley algorithm or Chart parsing algorithms. Disambiguation may alternatively or additionally be performed by comparison to representations of syntactic structures of similar phrases as detected using vector similarity, by reference to machine-learning algorithms and/or modules, or the like.

Still referring to FIG. 1, parsing module 112 may combine separately analyzed elements from at least a user input together to form a single query; elements may include words, phrases, sentences, or the like, as described above. For instance, two elements may have closely related meanings as detected using vector similarity or the like; as a further non-limiting example, a first element may be determined to modify and/or have a syntactic dependency on a second element, using dependency analysis or similar processes as described above. Combination into a query may include, without limitation, concatenation. Alternatively, or additionally, parsing module 112 may detect two or more queries in a single user input of at least a user input; for instance, parsing module 112 may extract a conversational query and an informational query from a single user input. An informational query, as used in this disclosure, is a query used to retrieve one or more elements of factual information; one or more elements may include, without limitation, any data suitable for use as a prognostic label, an ameliorative process label, and/or biological extraction data as described above. One or more elements may include an identity of a category of a prognostic label, ameliorative process label, biological extraction datum, informed advisor, or the like. One or more elements may include an identity of any factual element, including an identity of a place, person, informed advisor, user, entity, or the like. A conversational query, as used herein, is a query used to generate a textual response and/or response form, such as an overall sentence structure, templates, words, and/or phrases such as those usable for entries in narrative language database as described above, for inclusion of information returned in response to an informational query, for a response to a question, comment, phrase, or sentence that is not in itself a request for information, and/or for a request for clarification and/or more information as described in further detail below. A conversational query may include one or more pattern-matching elements, such as regular expressions, “wildcards,” or the like.

With continued reference to FIG. 1, parsing module 112 may be configured to convert at least a query into at least a canonical or standard form of query; for instance, and without limitation, once a query has been detected, parsing module 112 may convert it to a highly closely related query based on vector similarity, where the highly closely related query is labeled as a standard form or canonical query. In an embodiment, converting to a standard form query may enable more efficient processing of queries as described below, as a reduced space of potential queries may be used to retrieve conversational and/or informational responses.

Still referring to FIG. 1, computing device 104 is further configured to generate the at least a symptom datum as a function of the at least a query. In an embodiment, the at least a symptom datum may be generated as a function of the at least a query by the language processing module 116. Language processing module 116 may be involved in textual analysis and/or generation of text at any other point in machine-learning and/or communication processes undergone by the computing device 104.

Still referencing FIG. 1, computing device 104 is configured to train a machine learning process 120 with an expert input training set 124 from an expert knowledge database 128, wherein the expert input training set 124 further comprises prognostic data correlated to causal link data. A “causal link” as used in this disclosure, includes a descriptor containing a root cause correlated to a prognosis. A “root cause” as used in this disclosure, includes an identifier as to why auser has a particular prognosis and what can be done to restore function. Root cause may include an analysis of deeper causes of particular medical conditions and symptoms. For instance and without limitation, a prognosis such as fibromyalgia may have a root cause that includes toxin exposure while a prognosis such as hypothyroidism may be due to digestive inflammation. Root cause may include a functional medicine centric approach that may use evidence-based approaches to reverse chronic illness. For instance and without limitation, a prognosis such as frontal headache may be correlated to a causal link such as magnesium deficiency while a prognosis such as abdominal bloating may be correlated to a causal link such as small intestinal bacterial overgrowth (SIBO). In an embodiment, a prognosis may be correlated to one or more casual links. For instance and without limitation, a prognosis such as endometriosis may be correlated to a first causal link such as progesterone deficiency and a second causal link such as impaired estrogen detoxification. Expert input may provide relative statistics and likelihoods of a prognosis being correlated to a particular causal link. For instance and without limitation, a prognosis such as hypothyroidism may be correlated to a first causal link such as Hashimoto's thyroiditis that includes a sixty five percent likelihood and a second causal link such as impaired conversion of thyroxine (T4) to triiodothyronine (T3) that includes a twenty five percent likelihood.

With continued reference to FIG. 1, an “expert input training set” as used in this disclosure, includes expert submissions from expert authorities describing prognostic data correlated to causal link data. Expert authorities may include functional medicine health professionals such as doctors, nurse practitioners, physician assistants, and the like who may practice a particular sect of functional medicine and who may be considered a leading authority in his or her field of expertise. Expert authorities may have particular credentials, training and/or experience to be considered an expert in a field. For example, expert authorities may include experts who have certifications issued by THE INSTITUTE FOR FUNCTIONAL MEDICINE of Federal Way, Wash. In yet another non-limiting example, expert authorities may include experts who have certifications issued by AMERICAN ACADEMY OF ANTI-AGING MEDICINE (A4M) OF Boca Raton, Fla. Expert submissions may include datasets describing particular diagnoses and known causal links or root causes of diagnoses. Root cause of disease includes a holistic approach that addresses the underlying cause of a disease or diagnosis as opposed to a particular treating a symptom of a disease.For instance and without limitation, an expert submission may describe a blocked artery as having a root cause of high inflammation. In yet another non-limiting example, an expert submission may describe a headache experienced repeatedly before menstruation as having a root cause of low progesterone.

Still referring to FIG. 1, expert submissions may be entered by an expert and stored within expert knowledge database 128. Expert knowledge database 128 may include any data structure suitable for use as machine-learning database 132. Expert knowledge database 128 may store and/or organize expert submissions. In an embodiment, expert knowledge database 128 may store expert submissions by prognosis and/or causal link data as described in more detail below. Expert submissions may include textual entries from journals and/or research papers as described in more detail below.

With continued reference to FIG. 1, expert submissions may be entered into expert knowledge database 128 by an expert using an advisor client device 136. Advisor client device 136 may include any device suitable for use as a user client device 108 as described above. In one embodiment, the advisor client device 136 may include a graphical user interface whereby an expert may enter expert inputs. Graphical user interface may include any of the graphical user interfaces suitable for use on user client device 108 as described above. In an embodiment, advisor client device 136 may include a graphical user interface whereby an expert may enter expert inputs. Graphical user interface may include free form textual inputs and/or a drop-down menu whereby an expert may select an option.

Still referring to FIG. 1, machine-learning process 120 as described in this disclosure may be used to generate machine-learning models. A “machine-learning model,” as used in this disclosure, is a mathematical representation of a relationship between inputs and outputs, as generated using any machine-learning process including without limitation any process as described above, and stored in memory; an input is submitted to a machine-learning model once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model may be generated by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning, for instance for multi-layered networks.

Alternatively, or additionally, and still referring to FIG. 1, training data may include one or more elements that are not categorized; that is, training data may not be formatted or contain descriptors for some elements of data. “Training data,” as used in this disclosure, is data containing correlation that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to variouscorrelations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training datamay include data entered in standardized forms by persons or processes, such that entry of a givendata element in a given field in a form may be mapped to one or more descriptors of categories. Elements in training data may be linked to descriptors of categories by tags, tokens, or other data elements; for instance, and without limitation, training data may be provided in fixed-length formats,formats linking positions of data to categories such as comma-separated value (CSV) formats and/orself-describing formats such as extensible markup language (XML), enabling processes or devices todetect categories of data. Machine-learning algorithms and/or other processes may sorttraining data according to one or more categorizations using, for instance, processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below. Training data used by at least a server may correlate any input data as described in this disclosure to any output data as described in this disclosure.

Additionally, or alternatively, training data may be stored in a machine learning database 132. Machine-learning database 132 may include any data structure suitable for ordered storage and retrieval of data, which may be implemented as a hardware or software module. Machine-learning database 132 may be implemented, without limitation, as a relational database, a key-value retrieval datastore such as a NoSQL database, or any other format or structure for use as a datastore that a person skilled in the art would recognize as suitable upon review of the entirety of this disclosure.

Continuing to refer to FIG. 1, the computing device 104 is configured to assign weights, as a function of the at least a symptom datum, to the correlated data. In one embodiment, a weighted correlation may be used. In one nonlimiting example, the weights assigned may be the percentage of the correlated data that is present in the expert input training set 124 as a function of the symptom datum. In a nonlimiting example, correlations of one symptom in the at least a symptom datum may receive a higher weight than correlations of another symptom also present in the at least a symptom datum.

Alternatively, or additionally, the computing device 104 may be further configured to assign weights to the correlated data as a function of the prognostic labels.

Still referring to FIG. 1, the computing device 104 is further configured to identify root causes as a function of the assigned weights. In a nonlimiting example, the computing device may identify a root cause as a function of the highest positive correlation of the at a least a symptom datum to the causal links in the expert input training set. In another nonlimiting example, computing device 104 will identify root causes as a function of the number of positive correlations between an at least a symptom datum and the causal links in the expert input training set 124.

Still referring to FIG. 1. In an embodiment, computing device 104 is configured to correlate the at least a user symptom to the identified root causes as a function of the machine learning process 120. A plurality of methods may be used to correlate the at least a symptom datum to the expert input training set such as Pearson correlation, Kendall rank correlation, Spearman correlation, Point-Biserial correlation, and the like.

Alternatively, or additionally, computing device 104 is further configured to correlated prognostic labels to the identified root causes as a function of the machine learning process 120.

Alternatively, or additionally, the computing device 104 is further configured to identify a causal link as a function of the assigned weights. The computing device 104 may be further configured to transmit the causal link to an advisor client device 136.

With continued reference to FIG. 1, computing device 104 is configured to display the root causes to the user. In one embodiment, root causes may be displayed through a GUI in the user client device 108. In one embodiment, root causes may be displayed through an automated voice recording. In another embodiment, root causes may be transmitted to an advisor client device 136.

Alternatively, or additionally, the computing device 104 may be further configured to transmit the root cause to an advisor client device 136.

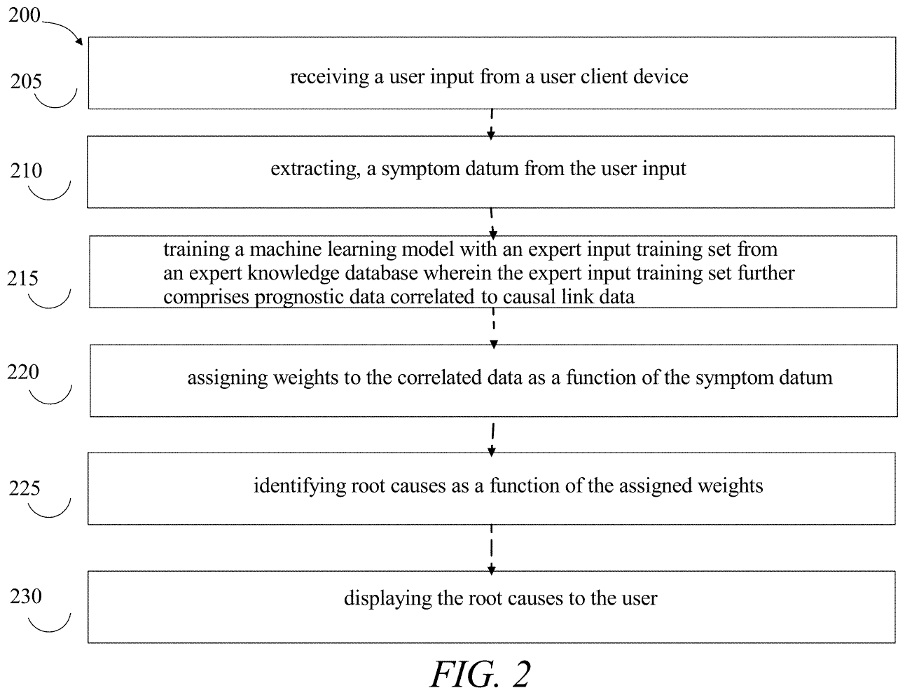

Referring now to FIG. 2, an exemplary embodiment of a method 200 of identifying a root cause is illustrated. At step 205, the method 200 comprises of receiving, by the computing device 104, a user input from a user device 108.

Still referring to FIG. 2, at step 210, the method 200 comprises extracting, by the computing device 104, at least a symptom datum from the user input. Where extracting the at least a symptom datum includes generating at least a query using the user input, and generating the at least a textual output as a function of the at least a query.

With continued reference to FIG. 2, at step 215, the method 200 comprises training, by the computing device 104, a machine learning model with an expert input training set 124 from an expert knowledge database wherein the expert input training set 124 further comprises prognostic data correlated to causal link data.

Continuing to refer to FIG. 2, at step 220, the method 200 comprises assigning weights, by the computing device 104, to the correlated data as a function of the at least a symptom datum.

Still referring to FIG. 2, at step 225, the method 200 comprises identifying, by the computing device 104, root causes as a function of the assigned weights. In one embodiment, method 200 may include correlating, by the computing device 104, the symptom datum to the root causes as a function of the machine learning process 120

With continued reference to FIG. 2, at step 230, the method 200 comprises displaying, by the computing device 104, the root causes to the user.

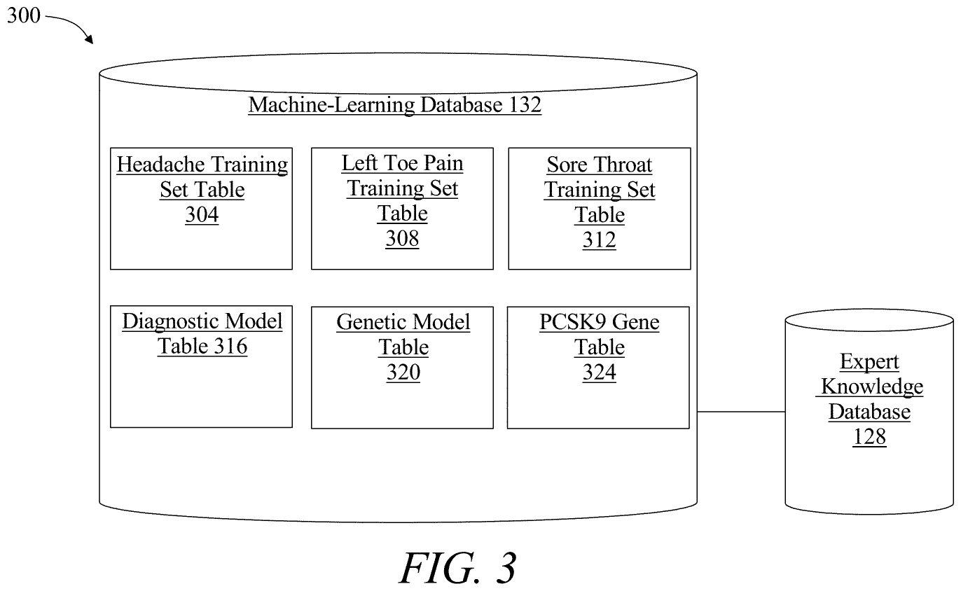

Referring now to FIG. 3, an exemplary embodiment of machine-learning database 132 300 is illustrated. Machine-learning database 132 may include any data structure for ordered storage and retrieval of data, which may be implemented as a hardware or software module, and which may be implemented as any database structure suitable for use as machine-learning database 132 as described above in FIG. 1. One or more tables contained within machine-learning database 132 may include headache training set table 304; headache training set table 304 may include one or more data entries containing symptom data that includes a symptom such as headache correlated to prognostic data. One or more tables contained within machine-learning database 132 may include left toe pain table 308; left toe pain table 308 may include one or more data entries containing symptom data that includes a symptom such as left toe pain correlated to prognostic data. One or more tables contained within machine-learning database 132 may include sore throat table 312; sore throat table 312 may include one or more data entries containing symptom data that includes sore throat correlated to prognostic data. One or more tables contained within machine-learning database 132 may include diagnostic model table 316; diagnostic model table 316 may include one or more diagnostic models that may be utilized to generate a supervised machine-learning process. One or more tables contained within machine-learning database 132 may include genetic model table 320; genetic model table 320 may include one or more genetic models that may be utilized to generate a supervised machine-learning process. One or more tables contained within machine-learning database 132 may include proprotein convertase subtilisin kexin type 9 (PCSK9) table 324; PCSK9 table may include one or more data entries containing genetic data such as PCSK9 sequence correlated to prognostic data.

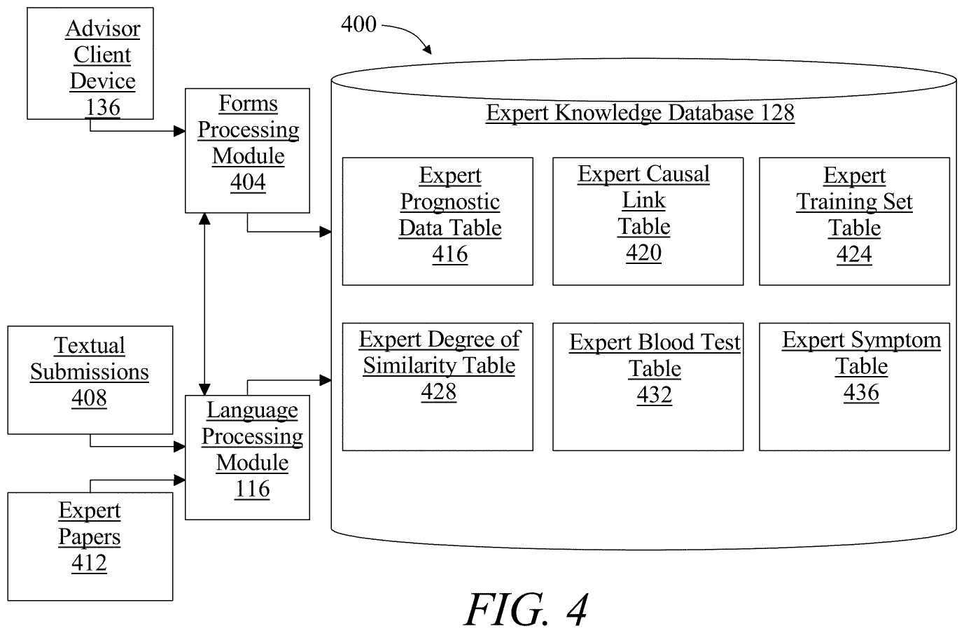

Referring now to FIG. 4, an exemplary embodiment 400 of expert knowledge database 128 is illustrated. Expert knowledge database 128 may include any data structure for ordered storage and retrieval of data, which may be implemented as a hardware or software module, and which may be implemented as any database structure suitable for use as machine-learning database 132. Expert knowledge database 128 includes a forms processing module 404 that may sort data entered in a submission via a GUI in an advisor client device 136 by, for instance, sorting data from entries in the advisor client device 136 to related categories of data; for instance, data entered in an entry, entered in the advisor client device 136, relating to a symptom training data set may be sorted into variables and/or data structures for storage of symptom training data sets, while data entered in an entry relating to a symptom training set may be sorted into variables and/or data structures for the storage of, respectively, categories of symptom training data. Where data is chosen by an expert from pre-selected entries such as drop-down lists, data may be stored directly; where data is entered in textual form, language processing module 116 may be used to map data to an appropriate existing label, for instance using a vector similarity test or other synonym-sensitive language processing test to map physiological data to an existing label. Alternatively or additionally, when a language processing algorithm, such as vector similarity comparison, indicates that an entry is not a synonym of an existing label, language processing module 116 may indicate that entry should be treated as relating to a new label; this may be determined by, e.g., comparison to a threshold number of cosine similarity and/or other geometric measures of vector similarity of the entered text to a nearest existent label, and determination that a degree of similarity falls below the threshold number and/or a degree of dissimilarity falls above the threshold number. Data from expert textual submissions 408, such as accomplished by filling out a paper or PDF form and/or submitting narrative information, may likewise be processed using language processing module 116. Data maybe extracted from expert papers 412, which may include without limitation publications in medical and/or scientific journals, by language processing module 116 via any suitable process as described herein. Persons skilled in the art, upon reviewing the entirety of this disclosure, will be aware of various additional methods whereby novel terms may be separated from already-classified terms and/or synonyms therefore, as consistent with this disclosure.

With continued reference to FIG. 4, one or more tables contained within expert knowledge database 128 may include expert prognosis table 416; expert prognosis table 416 may include any information provided by one or more experts regarding prognoses. One or more tables contained within expert knowledge database 128 may include expert causal link table 420; expert causal link table 420 may include any information provided by one or more experts regarding causal links. One or more tables contained within expert knowledge database 128 may include expert training set table 424; expert training set table 424 may include any information provided by one or more experts regarding training sets including diagnostic training sets and/or genetic training sets. One or more tables contained within expert knowledge database 128 may include expert degree of similarity index table 428; expert degree of similarity index table 428 may include any information provided by one or more experts regarding degree of similarity. One or more tablescontained within expert knowledge database 128 may include expert blood test table 432; expert blood test table 432 may include any information provided by one or more experts regarding bloodtest data. One or more tables contained within expert knowledge database 136 may include expertsymptom table 436; expert symptom table 436 may include any information provided by one or more experts regarding symptom data.

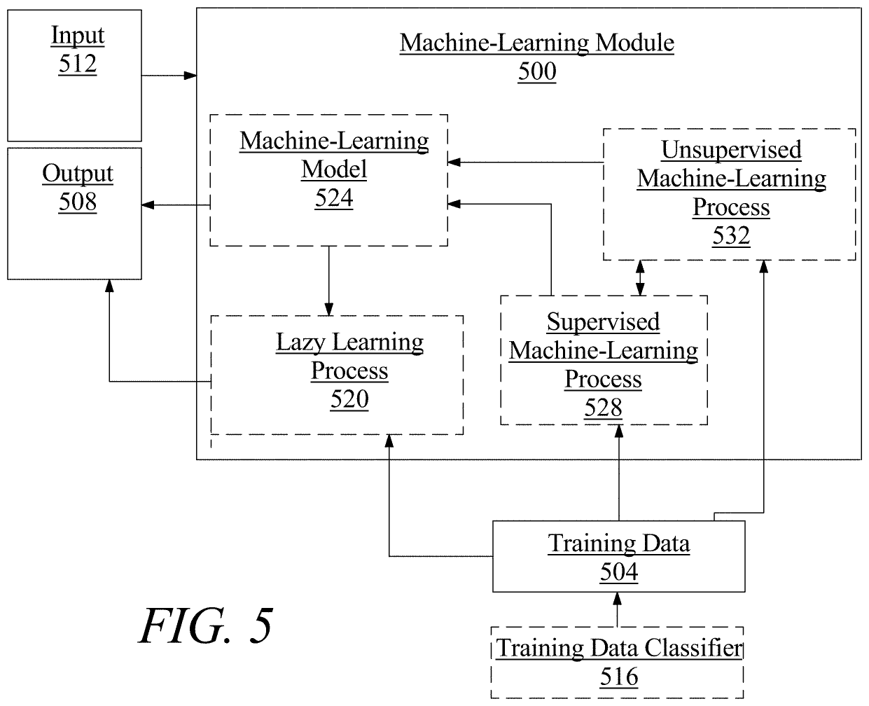

Referring now to FIG. 5 an exemplary embodiment of a machine-learning module 500 that may perform one or more machine-learning processes as described in this disclosure is illustrated. Machine-learning module may include any suitable Machine-learning module may perform determinations, classification, and/or analysis steps, methods, processes, or the like as described in this disclosure using machine learning processes. A “machine learning process,” as used in this disclosure, is a process that automatedly uses training data 504 to generate an algorithm that will be performed by a computing device/module to produce outputs 508 given data provided as inputs 512; this is in contrast to a non-machine learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, each entry representing a set of data elements that were recorded, received, and/or generated together; data elements may be correlated by shared existence in a given data entry, by proximity in a given data entry, or the like. Training data 504 may correspond to at least an element of data entry that may be used for training, a subset of a training data 504, and/or multiple training data sets XX04. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a nonlimiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example symptom datum input data and correlated data outputs determined from training data that relates biotic extraction 108 data to ranges of numerical values that may be used to determine a root cause.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 500 may generate a classifier using a classification algorithm, defined as a process whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to sections of data as it relates to subsets of users and the corresponding numerical values that result in the correlated data.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine learning algorithms as described in further detail below.

Alternatively or additionally, and with continued reference to FIG. 5, machine-learning processes as described in this disclosure may be used to generate machine-learning model 524. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. Supervised machine-learning processes may include any of the supervised machine-learning processes as described above in reference to FIG. 1. Supervised machine-learning processes include algorithms that receive a training set relating a number of inputs to a number of outputs and seek to find one or more mathematical relationships between inputs to outputs, where each of the one or more mathematical relations is optimal according to some criterion specified to the algorithm using some scoring function. For instance, a supervised learning algorithm may use elements of symptom data as inputs, prognoses as outputs, and a scoring function representing a desired form of relationship to be detected between elements of symptom data and prognoses; scoring function may, for instance, seek to maximize the probability that a given element of symptom data and/or combination of elements of symptom data is not associated with a given prognosis and/or combination of prognoses. Scoring function may be expressed as a risk function representing an “expected loss” of an algorithm relating inputs to outputs, where loss is computed as an error function representing a degree to which a prediction generated by the relation is incorrect when compared to a given input-output pair provided in symptom training data. Persons skilled in the art, upon reviewing the entirety of this disclosure, will be aware of various possible variations of supervised machine-learning algorithms that may be used to determine relation between elements of symptom data and prognoses. In an embodiment, one or more supervised machine-learning algorithms may be restricted to a particular domain for instance, a supervised machine-learning process may be performed with respect to a given set of parameters and/or categories of parameters that have been suspected to be related to a given set of symptom data, and/or are specified as linked to a medical specialty and/or field of medicine covering a particular set of symptoms. As a non-limiting example, a particular set of prognoses may be linked to particular symptoms and a supervised machine-learning process may be performed to relate symptoms to prognoses; in an embodiment, domain restrictions of supervised machine-learning procedures may improve accuracy of resulting models by ignoring artifacts in training data. Domain restrictions may be suggested by experts and/or deduced from known purposes for particular evaluations and/or known treatments for particular diseases and/or stages of disease. Additional supervised learning processes may be performed without domain restrictions to detect, for instance, previously unknown and/or unsuspected relationships between symptom data and prognostic data. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

Further referring to FIG. 5, machine learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic, or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminate analysis. Machine-learning algorithms may include kernel ridge regression. Machine learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 5, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data 504.

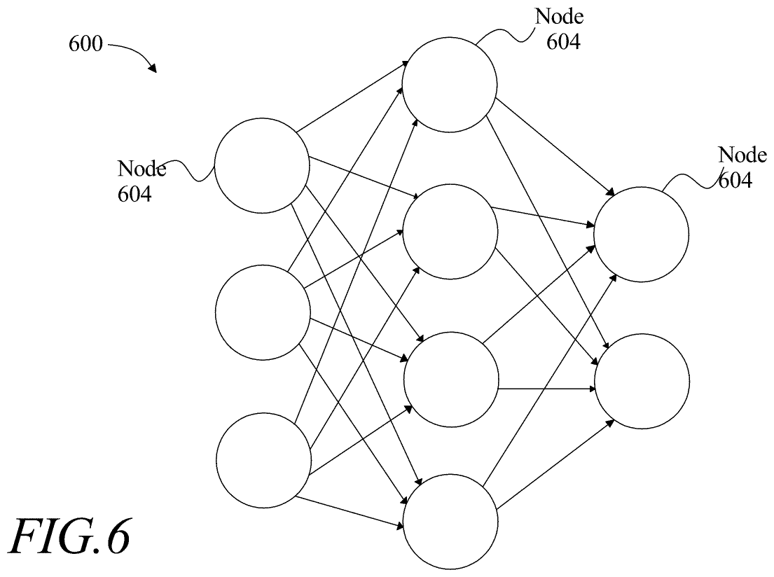

Referring now to FIG. 6, an exemplary embodiment of neural network 600 is illustrated. A neural network also known as an artificial neural network, is a network of “nodes,” or data structures having one or more inputs, one or more outputs, and a function determining outputs based on inputs. Such nodes 604 may be organized in a network, such as without limitation a convolutional neural network, including an input layer of nodes 604, one or more intermediate layers, and an output layer of nodes 604. Connections between nodes 604 may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

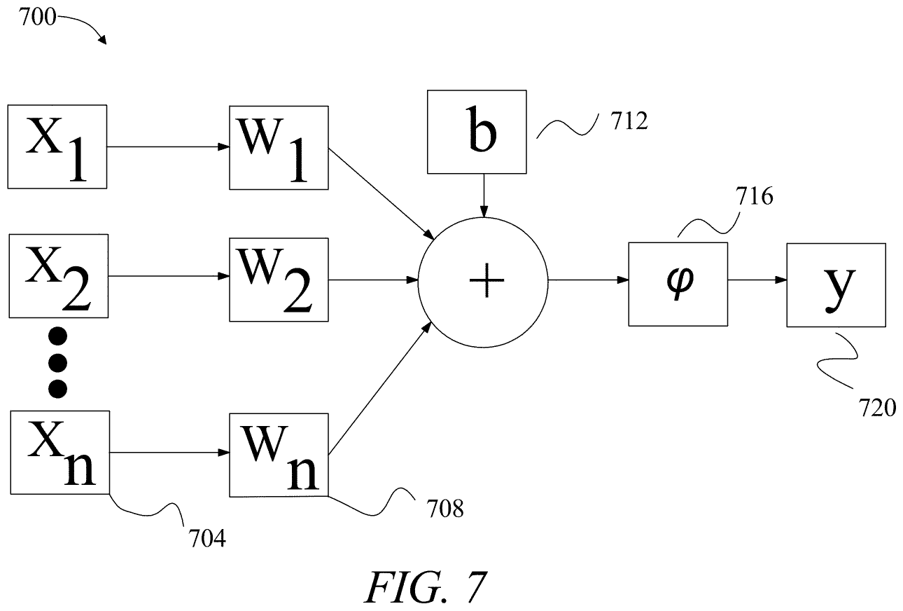

Referring now to FIG. 7, an exemplary embodiment of a node 700 of a neural network 800 is illustrated. A node may include, without limitation a plurality of inputs xₙ 704 that may receive numerical values from inputs to a neural network containing the node and/or from other nodes. Node may perform a weighted sum of inputs using weights wₙ 708 that are multiplied by respective inputs xₙ 704. Additionally or alternatively, a bias b 712 may be added to the weighted sum of the inputs such that an offset is added to each unit in the neural network layer that is independent of the input to the layer. The weighted sum may then be input into a function φ 716, which may generate one or more outputs y 720. Weight wₙ 708 applied to an input xₙ 704 may indicate whether the input is “excitatory,” indicating that it has strong influence on the one or more outputs y, for instance by the corresponding weight having a large numerical value, and/or a “inhibitory,” indicating it has a weak effect influence on the one more inputs y 720, for instance by the corresponding weight having a small numerical value. The values of weights wₙ 708 may be determined by training a neural network using training data, which may be performed using any suitable process as described above. In an embodiment, and without limitation, a neural network may receive semantic units as inputs and output vectors representing such semantic units according to weights wn that are derived using machine-learning processes as described in this disclosure.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

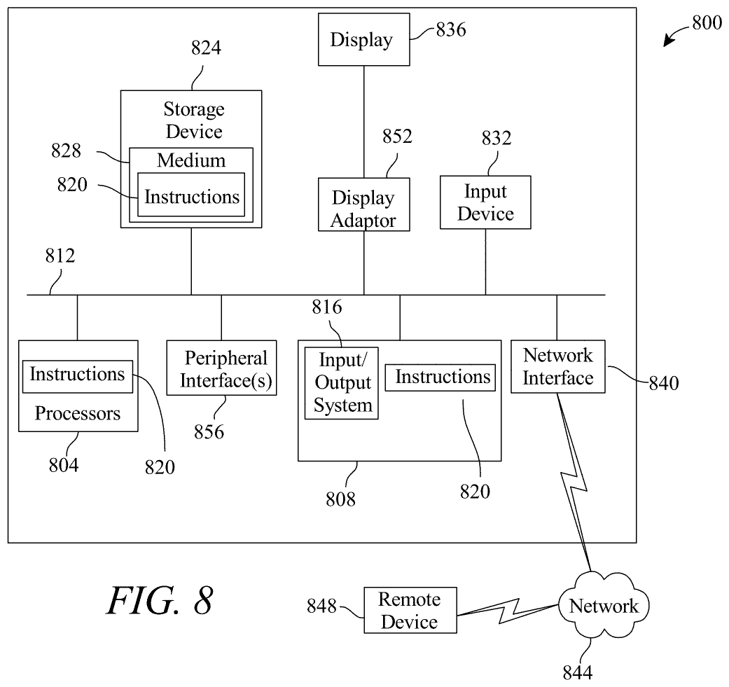

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for identifying a root cause, the system comprising a computing device, wherein
the computing device is designed and configured to:
receive a user input from a user client device;
extract at least a symptom datum from the user input, wherein the extraction of the symptom datum comprises;
generate at least a query using the at least a user input; and
generate the at least a symptom datum as a function of the at least a query;
train a machine learning process with an expert input training set wherein the expert input training set further comprises prognostic data correlated to causal link data;
assign weights, as a function of the at least a symptom datum, to the correlated data;
identify root causes as a function of the assigned weights; and
display the root causes to the user.

2. The system of claim 1, wherein the extracting the symptom datum further comprises using natural language processing.

3. The system of claim 1, wherein the computing device is further configured to extract prognostic labels from the symptom datum.

4. The system of claim 1, wherein the computing device is further configured to correlate prognostic labels to the expert input training set as a function of the machine learning process.

5. The system of claim 4 wherein the computing device if further configured to assign weights to the correlated data as a function of the prognostic labels.

6. The system of claim 1, wherein the computing device is further configured to identify a causal link as a function of the assigned weights.

7. The system of claim 6, wherein the computing device is further configured to transmit the causal link to an advisor client device.

8. The system of claim 1, wherein the user input is a voice input.

9. The system of claim 1, wherein the computing device is configured to use neural networks to identify root causes as a function of the assigned weights.

10. The system of claim 1, wherein computing device is further configured to correlate the at least a symptom datum to the root causes as a function of the machine learning process.

11. A method of identifying a root cause, the method comprising:
receiving, by a computing device, a user input from a user client device;
extracting, by the computing device, a symptom datum from the user input wherein extracting the symptom datum comprises:
generating at least a query using the at least a user input; and
generating the at least a symptom datum as a function of the at least a query;
training, by the computing device, a machine learning model with an expert input training set from an expert knowledge database wherein the expert input training set further comprises prognostic data correlated to causal link data;
assigning weights, by the computing device, to the correlated data as a function of the at least a symptom datum;
identifying, by the computing device, root causes as a function of the assigned weights; and
displaying, by the computer device, the root causes to the user.

12. The method of claim 11, wherein the extracting the symptom datum further comprises using natural language processing.

13. The method of claim 11, wherein the method further comprises extracting, by the computing device, prognostic labels from the symptom datum.

14. The method of claim 11, wherein the method further comprises correlating, by the computing device, prognostic labels to the expert input training set as a function of the machine learning process.

15. The method of claim 14, wherein the method further comprises, by the computing device, assigning weights to the correlated data as a function of the prognostic labels.

16. The method of claim 11, wherein the method further comprises, by the computing device, identifying a causal link as a function of the assigned weights.

17. The method of claim 16, wherein the method further comprises, by the computing device, transmitting the causal link to an advisor client device.

18. The method of claim 11, wherein the user input is a voice input.

19. The method of claim 11, wherein identifying, by the computing device, root causes as a function of the assigned weights further comprises using neural networks.

20. The method of claim 11, wherein the method further comprises, by the computing device, correlating the at least a symptom datum to the root causes as a function of the machine learning process.
